(19) United States
(12) Patent Application Publication
(10) Pub. No.: US 2022/0126794 A1
(43) Pub. Date: Apr. 28, 2022

(54) Shopping Cart Washer

(71) Applicants: Ramey Atkins, Springcreek, NV (US); Steve Thunehorst, Springcreek, NV (US)

(72) Inventors: Ramey Atkins, Springcreek, NV (US); Steve Thunehorst, Springcreek, NV (US)

(21) Appl. No.: 17/081,989

(22) Filed: Oct. 27, 2020

(51) Int. Cl. B60S 3/04; B08B 3/02; B60S 3/00; A61L 2/07; A61L 2/22; A61L 2/26

(52) U.S. Cl. CPC B60S 3/04; B08B 3/022; B60S 3/002; A61L 2/07; A61L 2202/15; A61L 2/26; A61L 2202/17; A61L 2202/11; A61L 2/22

(57) ABSTRACT

The shopping cart washer is a structure enabled to automatically wash shopping carts, receiving dirty carts on one end, and outputting the cleaned cart on the other end. Said carts are transported through the structure by means of a moving conveyor belt. Towards the entrance of the structure are pipes that emit steam and strategically placed sprayers to squirt the carts with water or cleaning fluid. Towards the exit are cooling fans horizontally placed to blow on outgoing carts, cooling and drying them before removal from the conveyor belt.

### BACKGROUND

A shopping cart is a wheeled cart supplied by a shop or store, especially supermarkets, for use by customers inside the premises for transport of merchandise as they move around the premises, while shopping, prior to heading to the checkout counter, cashiers or tills. Increasing the amount of goods a shopper can collect increases the quantities they are likely to purchase in a single trip, boosting store profitability. Studies have shown that it is advisable for shoppers to sanitize the handles and basket areas prior to handling them or filling them with groceries due to high levels of bacteria that typically live on shopping carts. This is due to the carts having a high level of exposure to the skin flora of previous users. There have been no products available as original equipment or as an aftermarket to address this problem.

There are a multitude of means by which shopping carts may be infected or otherwise made dirty. Carts may be used to held sick children, raw meat can drip through packaging onto the cart, sick operators may spread germs onto the cart's handle, and babies may defecate on the cart. As such, shopping carts need to have a method to be sanitized between usages or on a frequent basis. There have been no products available as original equipment or as an aftermarket to address this problem either.

There exists a need for a shopping cart washer that is not being met by any known or disclosed device or system of present.

### SUMMARY OF THE INVENTION

The shopping cart washer is a structure enabled to automatically wash shopping carts, receiving dirty carts on one end, and outputting the cleaned cart on the other end. Said carts are transported through the structure by means of a moving conveyor belt. Towards the entrance of the structure are pipes that emit steam and strategically placed sprayers to squirt the carts with water or cleaning fluid. Towards the exit are cooling fans horizontally placed to blow on outgoing carts, cooling and drying them before removal from the conveyor belt.

### BRIEF DESCRIPTION OF THE DRAWINGS

FIG. 1 is a view of the shopping cart washer in accordance with an embodiment of the present disclosure.

FIG. 2 is a flow chart of a method of sanitizing shopping carts in accordance with an embodiment of the present disclosure.

FIG. 3 is a flow chart of further methods of sanitizing the shopping carts in accordance with an embodiment of the present disclosure

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Throughout the present disclosure the term ‘interleaved’ is used to refer to the undercarriage spray array disposed between halves or parts of the conveyance belt. The term spray array refers to a row and a column and any combination of rows and columns thereof array of spray nozzles or orifices for cleaning the shopping cart.

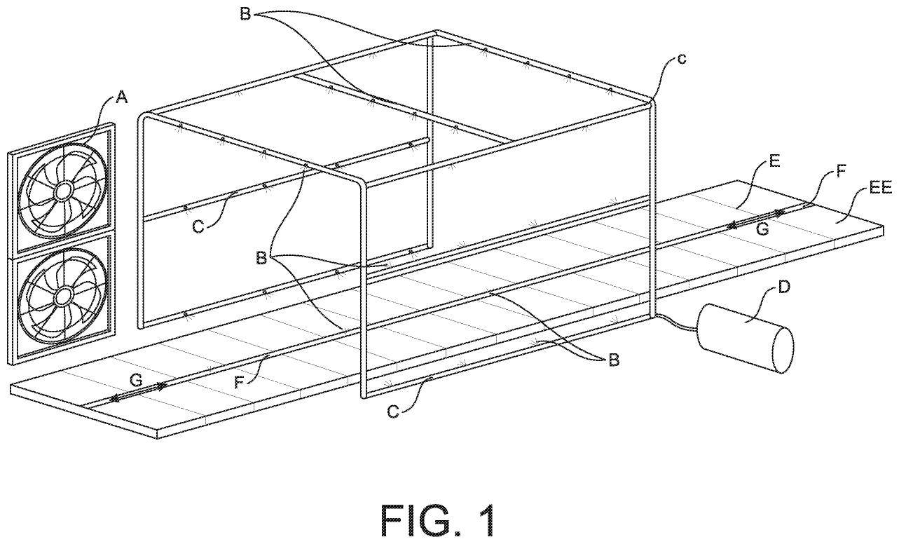

FIG. 1 is a view of the shopping cart washer in accordance with an embodiment of the present disclosure. Shopping carts are transported by means of the conveyor belt E. The carts are placed on the side depicted in the drawing on the right, and come out after having been sanitized on the left side. An external steamer D is connected to the pipework of steam pipes C which emits the steam upon the cart as it is cleaned. Additionally, strategically placed sprays B are in the shopping cart washer to clean the cart with a cleaning fluid or clean water. Before the carts are removed from the conveyor belt, they are cooled via the fans A. The undercarriage spray array F is disposed in a slot between two parts E and EE of the conveyance belt. The motion G indicated is a frontward and a backward conveyance via the indicated arrows. A controller and motion driver for predetermining conveyance motion, suspension and speed is not indicated but is included in the disclosure.

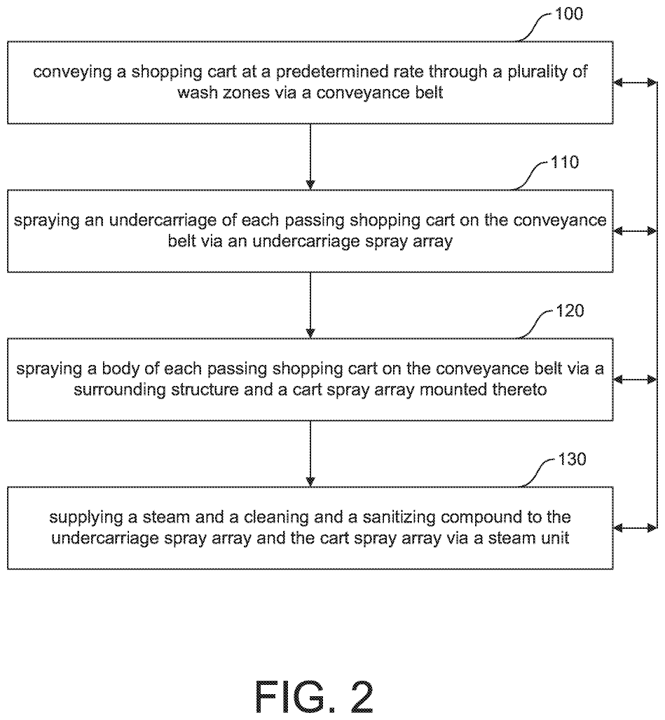

FIG. 2 is a flow chart of a method of sanitizing shopping carts in accordance with an embodiment of the present disclosure. The depicted method includes conveying 100 a shopping cart at a predetermined rate through a plurality of wash zones via a conveyance belt. The method also includes spraying 110 an undercarriage of each passing shopping cart on the conveyance belt via an undercarriage spray array. The method additionally includes spraying 120 a body of each passing shopping cart on the conveyance belt via a surrounding structure and a cart spray array mounted thereto. The method further includes supplying 130 a steam and a cleaning and a sanitizing compound to the undercarriage spray array and the cart spray array via a steam unit.

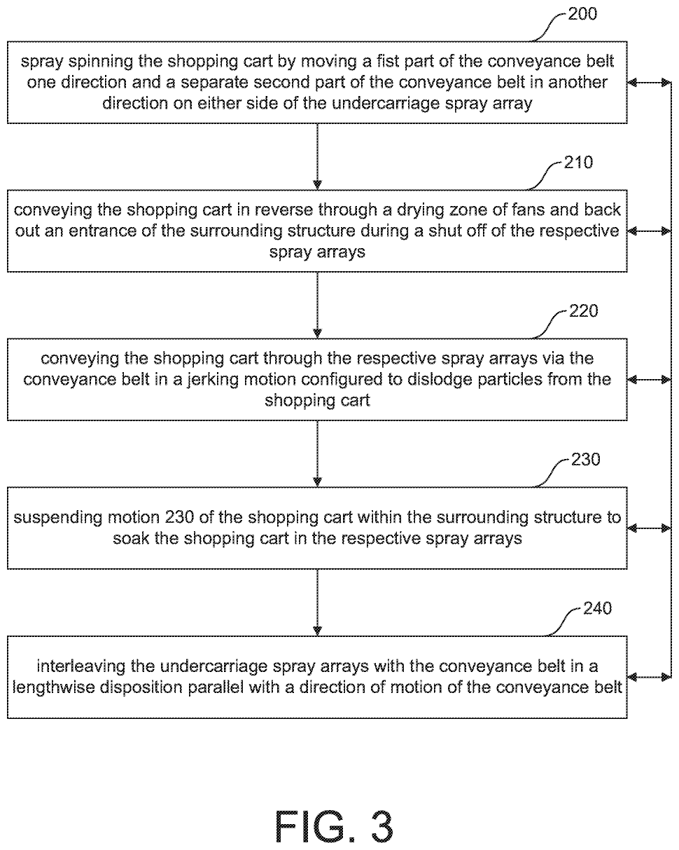

FIG. 3 is a flow chart of further methods of sanitizing the shopping carts in accordance with an embodiment of the present disclosure. Embodied methods include spray spinning 200 the shopping cart by moving a first part of the conveyance belt one direction and a separate second part of the conveyance belt in another direction on either side of the undercarriage spray array. The embodied methods also include conveying 210 the shopping cart in reverse through a drying zone of fans and back out an entrance of the surrounding structure during a shut off of the respective spray arrays. The methods additionally include conveying 220 the shopping cart through the respective spray arrays via the conveyance belt in a jerking motion configured to dislodge particles from the shopping cart. The methods further include suspending motion 230 of the shopping cart within the surrounding structure to soak the shopping cart in the respective spray arrays. The embodied methods yet include interleaving 240 the undercarriage spray arrays with the conveyance belt in a lengthwise disposition parallel with a direction of motion of the conveyance belt.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A shopping cart washing system comprising:
a conveyance belt configured to convey a shopping cart at a predetermined rate through a plurality of wash zones;
an undercarriage spray array configured to spray an undercarriage of each passing shopping cart on the conveyance belt;
a cart spray array mounted on a structure surrounding the shopping cart passing there through on the conveyance belt; and
a steam unit configured to supply a steam to the undercarriage spray array and the cart spray array and include a cleaning and sanitizing compound.

2. The system of claim 1, wherein the conveyance belt extends beyond the structure surrounding the cart in a direction of motion entering and leaving the structure.

3. The system of claim 1, wherein the conveyance belt moves the shopping cart to a threshold of the undercarriage and the cart spray arrays at a rate quicker than a rate it moves the shopping cart through the respective arrays.

4. The system of claim 1, wherein the conveyance belt take the shopping cart through the cart spray array in a jerking motion configured to dislodge particles from the cart.

5. The system of claim 1, wherein the conveyance belt suspends motion of the shopping cart within the surrounding structure to soak the shopping cart in the respective spray arrays.

6. The system of claim 1, wherein the undercarriage spray array is longitudinally interleaved with the conveyance belt in a floor of the system.

7. The system of claim 1, wherein the undercarriage spray array is configured with a plurality of spray nozzles which oscillate in a sweeping pattern onto an undercarriage of the shopping cart.

8. The system of claim 1, wherein the cart spray array is configured with a plurality of spray nozzles which oscillate in a sweeping pattern onto a body of the shopping cart.

9. The system of claim 1, wherein the steam unit supplies a pressurized steam to the respective spray arrays at a temperature above the boiling point of water.

10. The system of claim 1, wherein the stem unit supplies a pressurized steam and a cleaning and a sanitizing compound to the respective spray arrays.

11. The system of claim 1, further comprising an array of cooling and drying fans disposed at an egress of the shopping cart from the surrounding structure.

12. The system of claim 1, further comprising a drainage collection of the condensed steam and the cleaning and sanitizing compound.

13. The system of claim 1, wherein the surrounding structure comprises a steam and chemical distribution network for the respective spray arrays.

14. The system of claim 1, wherein the conveyance belt comprises a forward and a reverse conveyance through the surrounding structure for a round trip conveyance.

15. (canceled)

15. A method of sanitizing a shopping cart, the method comprising:
conveying a shopping cart at a predetermined rate through a plurality of wash zones via a conveyance belt;
spraying an undercarriage of each passing shopping cart on the conveyance belt via an undercarriage spray array;
spraying a body of each passing shopping cart on the conveyance belt via a surrounding structure and a cart spray array mounted thereto; and
supplying a steam and a cleaning and a sanitizing compound to the undercarriage spray array and the cart spray array via a steam unit.

16. The method of claim 15, further comprising spray spinning the shopping cart by moving a first part of the conveyance belt one direction and a separate second part of the conveyance belt in another direction on either side of the undercarriage spray array.

17. The method of claim 15, further comprising conveying the shopping cart in reverse through a drying zone of fans and back out an entrance of the surrounding structure during a shut off of the respective spray arrays.

18. The method of claim 15, further comprising conveying the shopping cart through the respective spray arrays via the conveyance belt in a jerking motion configured to dislodge particles from the shopping cart.

19. The method of claim 15, further comprising suspending motion of the shopping cart within the surrounding structure to soak the shopping cart in the respective spray arrays.

20. The method of claim 15, further comprising interleaving the undercarriage spray arrays with the conveyance belt in a lengthwise disposition parallel with a direction of motion of the conveyance belt.
